(19) United States
(12) Patent Application Publication
(10) Pub. No.: US 2010/0114313 A1
(43) Pub. Date: May 6, 2010

(54) ANTI-ADHESION SURGICAL MEMBRANE

(75) Inventors: Stéphane Lack, Beaucouze (FR); Michel Barikosky, Paris (FR); Christian Girardiere, Paris (FR)

Correspondence Address: J. Rodman Steele;Novak Druce & Quigg LLP, 525 Okeechobee Blvd, Suite 1500, West Palm Beach, FL, 33401, US

(73) Assignee: Les Laboratoires Brothier, Nanterre (FR)

(21) Appl. No.: 12/607,493

(22) Filed: Oct. 28, 2009

(30) Foreign Application Priority Data
Oct. 30, 2008 (FR) 0806055

(51) Int. Cl. A61F 2/02; A61B 17/03

(52) U.S. Cl. 623/11.11; 606/151

(57) ABSTRACT

The membrane is made of a polymeric material, characterized in that one (3) of its two faces at least has suppling and resorbability embossments (4).

This invention relates to the field of adhesions.

Adhesions are fibrous strips connecting normally isolated tissue surfaces. The formation of adhesions is a possible consequence of any surgery, as it is part of the healing process framework.

Adhesions occur in most patients having undergone a surgery in the abdomen or the pelvic area, as a hernia cure or a gynecologic or colorectal surgery. Such adhesions are also inherent to the tendon, heart surgery.

Adhesions could be at the origin of severe complications: occlusion of the small intestine, female sterility, chronic pains, problems upon subsequent surgeries, etc. Approximately three cases of intestine occlusion out of four and one case of chronic pelvic pain out of five, or even out of two, are caused by post-surgery adhesions. It often happens that new adhesions occur as a result of a surgery the aim thereof being precisely to eliminate the old ones, resulting, in addition to an additional surgery, in extending the healing time and increasing the risk, the cost and the complexity of the surgery.

This invention relates to an anti-adhesion surgical membrane to be thus used more particularly in digestive surgery for being interposed between the intestine and the abdomen wall. Generally speaking, this is to be used for preventing the mutual adhesion of tissues, for example, those of a tendon and the skin.

The anti-adhesion bioadhesive membranes could be obtained through water evaporation of a water solution and several percents of one or more polymers. Membranes are, as one might say, plasticized. As a polymer, an alginate ester, as for example the polyethylene glycol alginate, added with poloxamer and copper, could be contemplated.

The membranes being currently available on the market is not supple enough and the invention of the present application aims at increasing such supple feature.

Thus, this invention relates to an anti-adhesion surgical membrane in a polymeric material, characterized in that one of its two faces at least has suppling and resorbability embossments.

Embossments are referred to as suppling embossments as they are obtained upon manufacture by forming, providing anisotropy and increasing thereby the supple feature of the membrane.

Embossments are referred to as resorbability embossments, since in use, by means of the channels they form therebetween, they are able to retain the biological fluid that could incidentally act as a lubricant.

It is to be noticed that the embossments allow for the contact surface of the membrane to be decreased, thereby the latter can be moved more easily on its site, to position it correctly. In this respect, embossments could also be referred to as contact embossment.

It is further to be noticed that the embossments can enhance the resorption of the membrane of this invention, as its disappearance some time after healing is one of its qualities.

The material of the membrane of this invention could also comprise a cross-linking agent, an adjuvant, an active ingredient.

This invention will be better understood by reading the following description of an interesting embodiment of the membrane of this invention, in reference to the appended drawing, wherein:

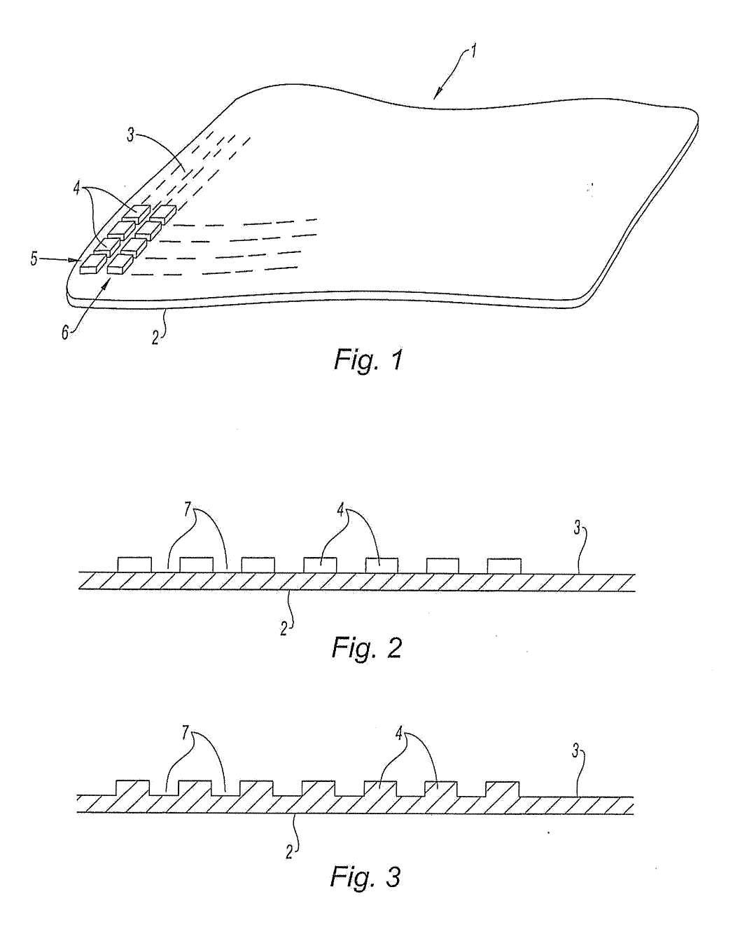

FIG. 1 is a top perspective view of the membrane;

FIG. 2 is a sectional view of the membrane, through a resorbability channel, and

FIG. 3 is a sectional view of the membrane through a row of plots running alongside a resorbability channel.

In order to form the membrane to be described, a mould part is first created with a bottom provided with small opened cells, being substantially parallelepipedic, regularly distributed at the surface of this bottom, here according to a matrix arrangement of rows and columns. It could be a conformer of the matrix stamp type.

A water and polymer solution is prepared. It is poured in the mould and the water is allowed to evaporate until a plasticized membrane 1 is obtained having, on one side, a planar surface 2 and, on the other side, a texturized or jagged face 3, with embossments or projections having the shape of small parallelepipedic plots 4, corresponding to the cells of the mould. The plots are distributed on the face 3 as a matrix made of rows 5 ad columns 6 running alongside resorbability channels 7, here being mutually orthogonal.

As a basic solution for forming the membrane, a solution of cross-linked propylene glycol alginate (alginate ester) has been contemplated, here added with a product of the registered trademark Lutrol®, glycerol (adjuvant) and copper, as a cross-linking ion. The polymer proportion in the contemplated species was 2% by volume. A proportion ranging from 0.5% to 10% polymer is appropriate. Such a range is appropriate for preventing the solution from being either too viscous or too aqueous.

As a cross-linking agent for alginate esters, one could use, generally, any multivalent ions, as precisely copper. Examples of an active ingredient include a vitamin, an anti-inflammatory agent, an antioxidant.

The addition of a vitamin complex (active ingredient) makes it possible to improve healing the tissues impaired upon surgery.

Thereby, suppling, resorbability and contact embossments are obtained.

Through the formation of embossments, the thus obtained membrane is provided with a high supple feature, it exhibits a valuable resorption ability and an easy repositioning. It could be packaged in rolls.

An anti-adhesion membrane has been described being provided with embossments only on one of its two faces. A membrane could be contemplated being texturized on both faces.

In the case where the membrane is only texturized on one of its faces, the other face could be adhesive.

Obviously, the membrane of this invention could be associated to a supple backing for use in some particular applications.

A membrane with parallelepipedic embossments has been described. Obviously, it could not be a limitative feature. The embossments could be frustoconical, rounded, cylindrical. The arrangement of the embossments could also be not as regular as a matrix arrangement.

1. An anti-adhesion surgical membrane in a polymeric material, characterized in that one (3) of its two faces at least has suppling and resorbability embossments (4).

2. A membrane according to claim 1, wherein said material comprises an alginate ester derivative.

3. A membrane according to claim 1, which has been formed through evaporation of a water solution of 0. 5 to 10% by volume of polymer.

4. A membrane according to claim 3, which has been formed through evaporation of the solution in a mould comprising cells corresponding to the embossments (4) of the membrane.
